(19) United States
(12) Patent Application Publication
(10) Pub. No.: US 2017/0298339 A1
(43) Pub. Date: Oct. 19, 2017

(54) Engineered Stable Microorganism/Cell Communities

(71) Applicants: David Hanson, Albuquerque, NM (US); Linnea K. Ista, Albuquerque, NM (US); Plamen B. Atanassov, Santa Fe, NM (US); John Michael Roesgen, Albuquerque, NM (US); Jose Cornejo, Albuquerque, NM (US)

(72) Inventors: David Hanson, Albuquerque, NM (US); Linnea K. Ista, Albuquerque, NM (US); Plamen B. Atanassov, Santa Fe, NM (US); John Michael Roesgen, Albuquerque, NM (US); Jose Cornejo, Albuquerque, NM (US)

(73) Assignee: STC.UNM, Albuquerque, NM (US)

(21) Appl. No.: 15/481,600

(22) Filed: Apr. 7, 2017

Related U.S. Application Data
(63) Continuation-in-part of application No. PCT/US15/54470, filed on Oct. 7, 2015.
(60) Provisional application No. 62/366,677, filed on Jul. 26, 2016.
(60) Provisional application No. 62/061,053, filed on Oct. 7, 2014.
(60) Provisional application No. 62/065,808, filed on Oct. 20, 2014.

(51) Int. Cl. C12N 11/04; C12N 1/20; C02F 3/32; C02F 3/00; C02F 3/34; H01M 8/16; C12N 1/12

(52) U.S. Cl. CPC C12N 11/04; H01M 8/16; C12N 1/20; C12N 1/12; C02F 3/005; C02F 3/341; C02F 3/325; C02F 3/322; C02F 2303/10

(57) ABSTRACT

Engineered stable multi-organism (or multi-cell type) communities encapsulated in a media that slows or prohibits certain metabolic functions such as cell division, but maintains other metabolic functions.

### CROSS-REFERENCE TO RELATED APPLICATIONS

The following application claims the benefit of U.S. Provisional Application No. 62/366,677 “Engineered Stable Microorganism communities,” filed Jul. 26, 2016 and is a Continuation-in-Part of PCT/US15/54470 “Engineered Stable Microorganism/Cell Communities” having an International Filing date of Oct. 7, 2015 which claims the benefit of U.S. Provisional Application Nos. 62/061,053, “Engineered, Modular Microbial Communities,” filed Oct. 7, 2014, and 62/065,808, “Engineered Stable Microorganism Communities,” filed Oct. 20, 2014, each of which is hereby incorporated by reference in its entirety.

### STATEMENT REGARDING GOVERNMENT SPONSORED RESEARCH

This invention was made with Government support under IIA-1301346 awarded by the National Science Foundation and grant #W911NF1210208 awarded by the Army Research Office (The U.S. Government has certain rights in this invention.

### BACKGROUND

Humans are not a single organism, but rather a complex super-organism that is a community of cells with more bacterial than human cells (10 bacterial cells for every 1 human cell). Oxygenic phototrophic eukaryotes are not really single organisms either, rather an obligate symbioses between two kinds of bacteria (mitochondria and plastids) and a host cell. There are countless other examples of symbiotic organisms and even communities, each arising by a combination of chance and selection where the function of the whole outperformed the sum of the parts. The ecological theory describing the increased stability and productivity has been well defined, and there are a handful of papers demonstrating how monocultures are out-performed by co-cultures of more than one species. Furthermore, the assembly of multiple cell types (multiple organisms) into a symbiotic super-organism community is only limited by the complexity of the system that can be created and managed.

Unfortunately, we have up until now, lacked the knowledge and tools to create a stable multi-species super-organism community. Attempts have been made with, for example, immobilization via alginate beads, but these miss the critical component of being stable. In the previously described systems, microorganisms were able to continuously grow and migrate throughout the immobilization media which meant that the community composition changed through time. However, rates of cell growth, division and production of metabolites have all been shown to increase in the alginate structures. These increases in cellular productivity occur when the cells are immobilized alone (Liu et al. 2012) but especially when co-immobilized with growth promoting bacteria such as Azospirillum (Gonzalez and Bashan 200, Gonzalez-Bashan et al 2000, Lebsky et al 2001, de-Bashan et al 2002).

Biofilms, that is communities of cells, have been encapsulated to improve energy production { Luckarift, 2010 #37}, bioremediation {Luckarift, 2011 #42} and wastewater treatment {Jaroch, 2011 #43}, but in all cases these have been monoculture communities lacking the complexity of interactions found in multispecies communities.{Connell, 2012 #67} While previous efforts in this regard have been the design of platforms on which to place individual cells or groups of cells, {Connell, 2012 #67} the idea of engineering the biofilm and preserving its naturally developed structure has not been addressed.

### SUMMARY

The present disclosure provides engineered stable multi-organism (or multi-cell type) communities encapsulated in a media that slows or prohibits certain metabolic functions such as cell division, but maintains other metabolic functions. According to an embodiment, these engineered multi-organism communities can be designed for the long-term, stable production of desired products including pharmaceuticals, amino acids, and electricity.

### BRIEF DESCRIPTION OF THE DRAWINGS

FIG. 1 depicts the metabolic differences between encapsulated and liquid cell culture populations.

FIG. 2 is a schematic illustration of a method for encapsulating organisms.

FIG. 3 compares graphs of photosynthetic O₂ production for liquid vs. encapsulated cultures of the microalga Chlorella sorokiniana.

FIG. 4 is another comparison of photosynthetic O₂ production for liquid vs. encapsulated cultures of the microalga Chlorella sorokiniana.

FIG. 5 is a comparison of the efficiency of photosynthetic electron transfer (PSII effective quantum yield) for liquid vs. encapsulated cultures the microalga Chlorella sorokiniana.

FIG. 6 compares graphs of photosynthetic O₂ production for liquid vs. encapsulated cultures of the cyanobacterium Synechocystis 6803.

FIG. 7 is another comparison of photosynthetic O₂ production for liquid vs. encapsulated cultures of the cyanobacterium Synechocystis 6803.

FIG. 8 is a comparison of the efficiency of photosynthetic electron transfer (PSII effective quantum yield) for liquid vs. encapsulated cultures of the cyanobacterium Synechocystis 6803.

FIG. 9 compares graphs of photosynthetic O₂ production for liquid vs. encapsulated co-cultures of the microalga Chlorella sorokiniana and the cyanobacterium Synechocystis 6803.

FIG. 10 is another comparison of photosynthetic O₂ production for liquid vs. encapsulated co-cultures of the microalga Chlorella sorokiniana and the cyanobacterium Synechocystis 6803.

FIG. 11 is a comparison of the efficiency of photosynthetic electron transfer (PSII effective quantum yield) for liquid and encapsulated co-cultures the microalga Chlorella sorokiniana of the cyanobacterium Synechocystis 6803.

FIG. 12 is a schematic illustration of an embodiment of an artificial biofilm suitable for use in a Microbial Fuel Cell (MFC).

FIG. 13 is a schematic illustration of another embodiment of an artificial biofilm suitable for use in a Microbial Fuel Cell (MFC).

FIG. 14 is a schematic illustration of yet another embodiment of an artificial biofilm suitable for use in a Microbial Fuel Cell (MFC).

### DETAILED DESCRIPTION

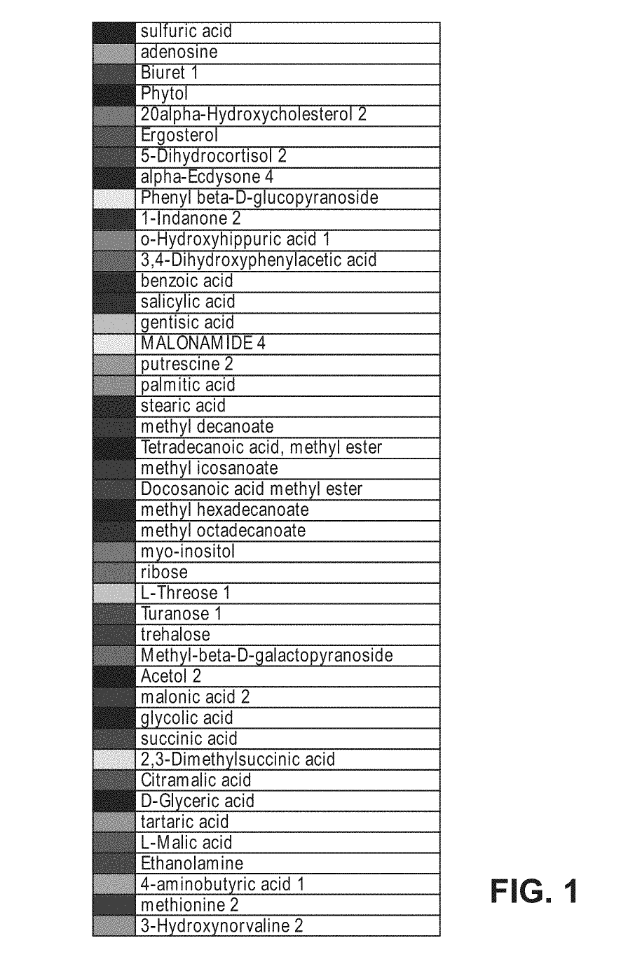

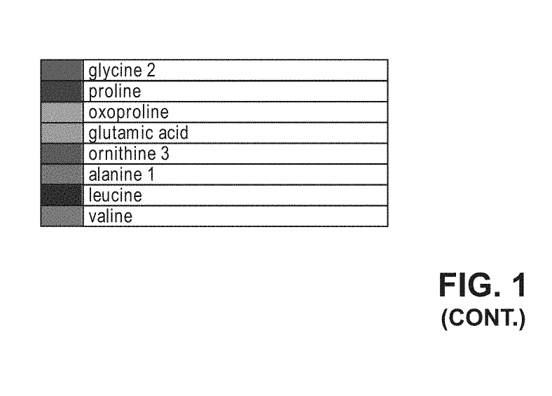

According to an embodiment the present disclosure provides engineered stable multi-organism communities encapsulated in a matrix that arrests certain phases of cell division or metabolic pathways, while enabling or even promoting others. For the purposes of the present disclosure the term “encapsulate” means the encapsulated organism is mechanically constrained within a matrix that the microorganism cannot itself degrade. In general, some, though not all, of the metabolic functions of an encapsulated microorganism are halted or arrested while others are maintained. For example, according to some embodiments, the encapsulation methods described herein limit growth and migration while maintaining biological function. Therefore, the initial community created can be stably maintained at (or near) the initially selected relative proportions (including both ratio and density) for long periods of time. For example, by using an imaging process to track individual encapsulated cells, we found that over a one-week period, a population of encapsulated algae increased in cell number by less than 5%, whereas parallel liquid cultures increased over 300%, and the metabolic profile was uniquely altered, as shown in FIG. 1, wherein compounds show with light colors next to them had lower concentrations than their liquid cultured equivalents and compounds shown with dark colors next to them had higher concentrations then their liquid cultured equivalents. Moreover, if the stable community then releases a compound of interest into the surrounding media, the product can be harvested continually for as long as biological function is maintained. Conversely, as long as there is a compound of interest in the surrounding media to be modified, converted, consumed, or destroyed by the community, this can occur continuously as controlled by the rate of supply to the surrounding media. The presently described communities can be stably maintained for months to years rather than the days to weeks that are seen in normal non-encapsulated cultures. Therefore, the cost of harvesting cultures, extracting products, and starting new cultures is greatly diminished.

According to an embodiment, the microorganism or cell community is created by encapsulated the microorganism or cells at desired ratios and densities in a silica-based encapsulation media. For the purposes of the present disclosure, the encapsulated microorganism and its encapsulating matrix or gel may be referred to as an “artificial biofilm.”

A variety of methods for encapsulating microorganisms or cells are known. One previously described method of encapsulating with silica has been used with biomolecules (Avnir et al 1994, Pierre 2004) and whole cells of everything from bacteria (Ferrer et al 2003, Fennouh et al 2000) to plants (Pressi et al 2003) to human cells and tissues (Pope et al 1997). Some of the original goals of encapsulation were to create chemical and biological sensors (Wang et al 2000), others include use in biofuel cells {Meunier, 2010 #23; Meunier, 2011 #20} and bioremediation {Al-Saraj, 1999 #28; Alvarez, 2011 #27}.

However, one of the drawbacks of encapsulation of whole cells is that previously described encapsulating processes have typically produced a toxic chemical as a byproduct. For example, two of the most common chemicals used to form silica sol-gels are Tetramethyl Orthosilicate (TMOS) and Tetraethyl Orthosilicate (TEOS) which go through condensation reactions producing methanol and ethanol as the respective byproducts, which can be harmful to biological species, (Dickson & Ely 2013).

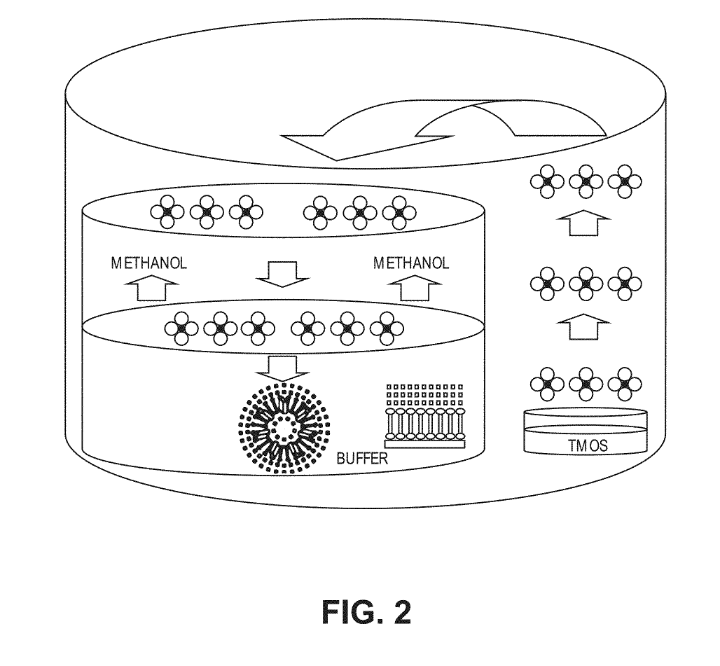

Accordingly it may be desirable to use an encapsulation method that does not utilize or which minimizes exposure of the cells/microorganism to harmful or toxic conditions. Thus, in a specific, non-limiting embodiment of the present disclosure, microorganisms are encapsulated using the techniques described in U.S. Pat. No. 8,252,607, titled “Bio-Compatible Hybrid Organic/Inorganic Gels: Vapor Phase Synthesis.” Briefly, and as shown in FIG. 2, an aqueous solution which may be, for example, a buffer containing the biological species to be encapsulated, is placed next to a vial containing a gel precursor in a closed container at a suitable temperature. Exemplary gel precursors including tetramethoxy silane (TMOS), tetraethoxy silane (TEOS), acrylic acid and other monomers, volatile organic or inorganic precursors such as metal alkoxy silanes, and metal chlorides such as Tic14, Sic14, etc. TMOS is relative volatile at 37° C., accordingly, 37° C. may be a suitable temperature for conducting the procedure when TMOS is used as a gel precursor. However, various temperatures may be used for a variety of different reasons. Under the aforementioned, or other suitable, conditions the gel precursor evaporates and is exposed to the buffer. According to various embodiments, the precursor may be delivered to the buffer by saturation, aerosol delivery, use of a nebulizer, ultrasonication, or other suitable means. Upon mixing with the buffer, the gel precursor is hydrolyzed. For example, TMOS hydrolyzes to silicon hydroxide and methanol. However, the relatively slow rate of transfer of the precursor leads to minimal methanol presence in the system at any given time, thus significantly reducing or even eliminating harmful effects to the biological species from the presence of methanol. Upon further condensation silicon dioxide is formed and leads to formation of a silica gel.

As stated above, the gels may be formed at 37° C., alternatively, the gels may be formed at other temperatures, including, for example, room temperature. In general, higher temperatures will result in a shorter gelation time and lower temperatures will result in a longer gelation time. According to some embodiments, the typical gelation time for room temperature synthesis of pH 7 buffer is around 6 hours. It will be appreciated that because the intended purpose is to encapsulate viable microorganisms or cells while maintaining functionality, the above-described methodology is well-suited, as the entire procedure can be carried out under conditions that are favorable to the microorganisms or cells. Accordingly, if a desired organism (or group of organisms) is known to thrive at a certain temperature range, the gelation method can be performed at that temperature range with the only change in the methodology being an appropriate increase or decrease in the gelation time.

Because the above-described method can easily be adjusted for compatibility with a with a wide variety of biologically suitable conditions, it should be understood that the present disclosure anticipates the encapsulation of a wide variety of organisms including, but not limited to, bacteria, single-celled algae, single celled fungi, viruses, suspensions of cells derived from multi-cellular organisms (e.g. plants and animals), and small multi-celled organisms such as lichens and colonial algae. In general, the organism to be encapsulated need only to be able to be suspended in a buffer with salt for the process to work.

It will also be appreciated that the methodology can easily be carried out using multiple types of microorganisms or cells, enabling the user to encapsulate multiple types of organisms or cells in the same gel. In general, the methodology can be utilized to immobilize any suspension of cells or organisms, whether it is a mixed- or mono-culture suspension. Moreover, the methodology can be applied to suspensions of pre-grown communities (naturally occurring or synthetic) or combined collections of pre-grown mixed- or mono-cultures, enabling very specific selection of the community membership. Furthermore, because the process typically results in very little die off after initial encapsulation, and the encapsulation generally prevents cell division and motility, the selected community membership will generally remain stable (in terms of both diversity and ratio) and metabolically active throughout the lifetime of the community.

In general, encapsulating a cell in a matrix of silica stops its growth and migration through the media, holding it somewhat suspended spatially. Accordingly, silica-based encapsulation matrices are typically designed to be rigid and chemically inert once formed. Although it will be understood that there may be an intervening temporary period when the cells are spatially suspended in a liquid gel and thus some of the methods described herein may take advantage of this liquid gel phase in order to form the final solid matrix.

According to various embodiments, entire communities of organisms can be encapsulated together. According to some embodiments, these communities may be self-contained ecosystems, able to maintain their existence without active intervention. According to other embodiments, nutrients may be added and/or products harvested. It should be appreciated that the term “product” is not necessarily limited to physical chemical or biological structures that are produced by one or more of the encapsulated organisms, but may include other tangible or intangible outputs or services such as heat, light, energy, clean water, sound, and aesthetically pleasing design. Accordingly, “harvesting” of these “products” is not necessarily limited to actual removal of a chemical or biological structure from the encapsulated community, but may also include the derivation of a benefit or change in condition due to the product. For example, if the encapsulated community is designed to produce heat, the “product” would be the heat and the act of “harvesting” that heat would simply be benefiting from the production of that heat or light. As a more specific example and as discussed in greater detail below, the encapsulated community could be engineered to enhance naturally or non-naturally occurring extracellular electron fluxes and/or transport, which could then be harnessed for electrical generation in, for example, a microbial fuel cell. In this case, “harvesting” of the electrons does not necessarily imply removal of the electrons (or product) from the system, but rather a harnessing of the process that takes place within the biofilm community for a benefit that is external to the community.

As stated above, the presently described methods enable the production of multi-organism communities wherein the communities can be specifically designed. However, this design is not limited to characteristics such as the relative ratio of one type of organism or cell type to another as described above. Other characteristics such as activity of metabolic pathways, density of cells in the matrix, physical and or chemical interaction between organisms and cells, physical proximity and position relative to each other, location relative to exchange surfaces with liquid media or gases and components within the liquid or gases, can all be designed and, importantly, maintained using the herein described system.

For example, the density of cells in the matrix can simply be controlled by controlling the ratio of cells in the cell suspension to the amount of gel precursor used. The higher the ratio of cells to gel precursor, the greater the density of cells in the matrix. Physical proximity and position can be controlled by direct positioning of organisms (e. g. laser entrapment and patterning of cells or printing of cells) or patterning chemical and/or physical features of the supporting substratum. During the matrix hardening process, cells often migrate due to biological (e.g. cell motility toward a stimulus) or physical (e.g. surface tension) effects, which can influence patterning like creating a ring of cells. Controlling the conditions during hardening can alter the pattern for desirable effects. Methods include but are not limited to adjusting salinity, pH, or matrix concentration to speed or slow hardening, mixing cells with a surfactant or an emulsifying agent (e.g. amorphous silica), or providing a directional stimulus (light, electromagnetic field, chemical attractant or repellent), or a stimulus that causes the cells to alter their surroundings (change surface chemistry, excrete compounds, etc.)

According to some embodiments, multiple gels encapsulating the same or different communities or monocultures can be layered or positioned relative to each other to form artificial biofilms comprising multiple modular communities. These artificial biofilms enable strict control of the community population as well as the interactions between the different modules, as desired. It will be appreciated that while the population of the communities can be strictly controlled, the present methodology does not require such control. Accordingly, communities with random or unknown diversity, ratios, population, etc., can be encapsulated using the exact same methodologies described above.

Layering or patterning can be achieved via multiple methods. These include, but are not limited to, successive gel formation from liquid mixtures, vapor deposition, mixing with agents that alter viscosity, and the use of molds or forms. Complex multi-dimensional structures can be easily made by mixing the TMOS/cell suspension with amorphous (fumed) silica prior to hardening such that it forms a paste with the appropriate viscosity to be extruded and maintain shape post-extrusion. This allows use of a device such as a three-dimensional printer to place the matrix in a pattern that is maintained as the matrix solidifies. According to a specific non-limiting example, encapsulated algae can be extruded through a syringe needle (using a screw drive and stepper motor attached to computer controlled mill) into a multi-layer structure resembling a grid when viewed from the top. This solidifies into an evenly green structure that is metabolically active as evidenced by the production of oxygen bubbles in the presence of light and CO₂.

According to a first embodiment, artificial biofilms can provide a platform to study the chemical or biological interactions between different communities/monocultures. For example, an encapsulated community formed from cells (or organisms) typically (or atypically) found in the gut could be placed in proximity to an encapsulated community formed from the cells (or organisms) typically (or atypically) found in the colon. One could then use this platform to study the chemical and biological interactions between the two communities and/or to study and research the response of these communities to different stimuli including, but not limited to, environmental conditions, and introduced naturally or non-naturally occurring compounds including pharmaceuticals, nutrients, etc.

Of course the use of multiple gels is not limited to research applications. In many cases, multiple layers of the same or different communities could be used to encourage, enable, or enhance the production of one or more desired products for industrial or other purposes. Other possible applications include the removal of undesirable compounds (natural or introduced), and providing or stimulating production of various pharmaceutical compounds. In addition, encapsulated communities found to have beneficial interactions could be ingested to relieve or treat ailments and/or assist in improving microbial communities inside an organism.

According to various embodiments the gels of the present disclosure can be incorporated into or themselves incorporate physical structures such as solid supports or matrices. These supports or matrices may father include one or more channels which may be used to supply nutrients to the encapsulated organisms or to remove products from the organisms' immediate environment. According to some embodiments, the encapsulated organisms may be positioned onto a substrate via spin-coating, patterning, layering, printing or any other suitable means including those described above.

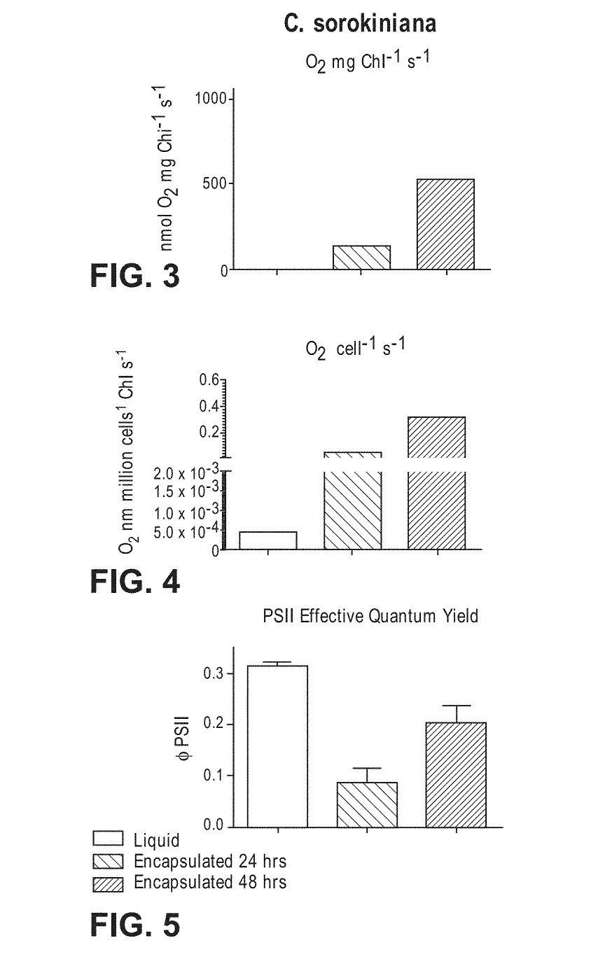

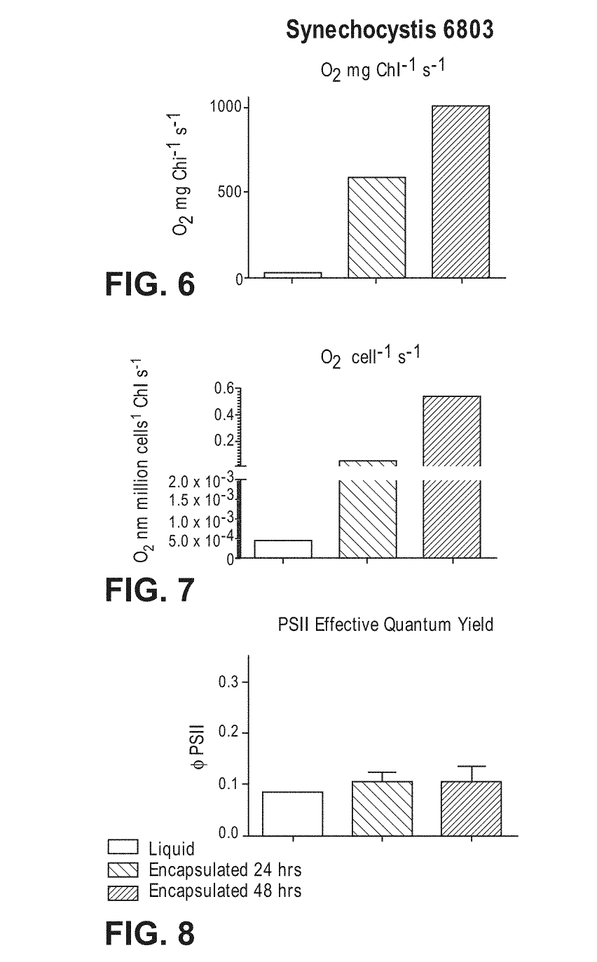

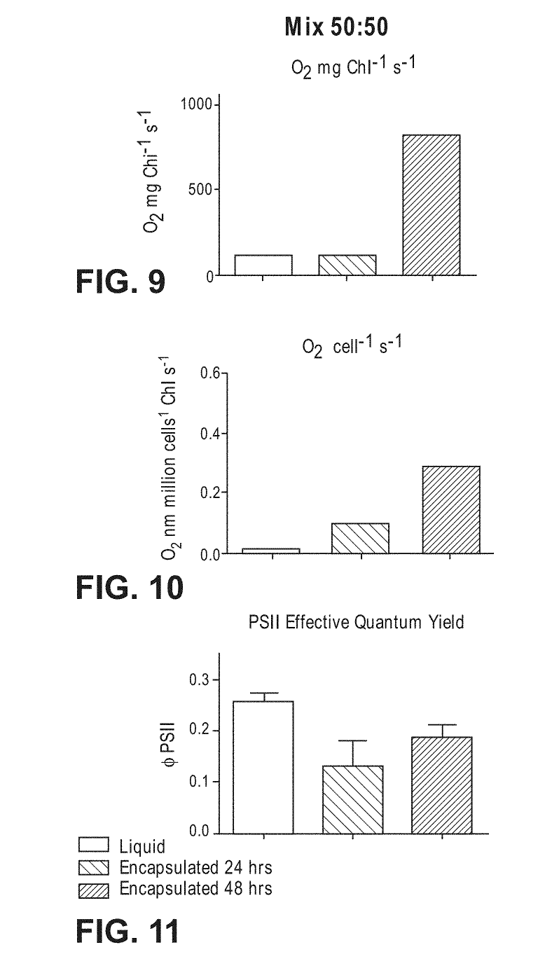

It will be appreciated that an important aspect of the encapsulation of the organisms as described herein is providing them with an environment that is conducive to the ultimate desired outcome. For example, it may be desirable that encapsulated photoautotrophic cells retain their photosynthetic capability. In this case, it may be desirable for the encapsulation media to be transparent in order to allow the cells to capture light for photosynthesis. In may also be desirable to modify the matrix to exclude or enhance the wavelengths of light reaching the cells. Exclusion could reduce damage from UV or other harmful wavelengths or excessive photon flux density Enhancement could increase the number of useable photons reaching cells at the surface and more deeply located in the matrix (for example, by improving light scattering or by providing micro-light channels). Similarly, CO₂ is also needed for photosynthesis either directly or through the supply and conversion of HCO₃⁻ and O₂ reduces carbon assimilation. Therefore, the matrix or community composition could also be modified to enhance CO₂ and HCO₃⁻ supply and diminish O₂, for example by including more heterotrophs that consume O₂ and generate CO₂. This same principle could maintain high or low O₂ for other cells in the matrix. For example, as shown in FIGS. 3-11, photosynthetic oxygen production of encapsulated cells was higher than that of liquid cultures for the microalga Chlorella sorokiniana, the cyanobacterium Synechocystis 6803 individually and when co-encapsulated, while the efficiency of photosynthetic electron transfer (PSII effective quantum yield) was maintained.

Accordingly, it will be appreciated that the artificial biofilms of the present disclosure can be useful for a wide variety of applications including, but not limited to, research applications, medical diagnostics and treatment, pharmaceutical production and removal, waste treatment, bio-electronics, and bio-energy including, but not limited to, microbial fuel cell applications.

As a specific example, the present disclosure provides a microbial fuel cell (MFC) that uses or incorporates an artificial biofilm encapsulating, for example, electrochemically active bacteria such as Shewanella oneidensis, Geobacter sulfurreducens, Shewanella putrefaciens or Aeromaonas hydrophila in the MFC's electrode. A typical microbial fuel cell includes an anode and a cathode separated by a cation specific membrane. Fuel is oxidized by microorganisms in the anode, generating CO₂, electrons and protons. The electrons are transferred to the cathode compartment through an external electric circuit, while the protons are transferred to the cathode compartment through the membrane. Electrons and protons are consumed in the cathode compartment, combining with oxygen to form water. This artificial biofilm may be formed from a single film (or module) or multiple films (or modules) layered or positioned in proximity to each other. For example, a first module may contain the electrochemically active bacteria while a second module may contain an encapsulated community that can produce fuel for the microorganisms, enhance the activity of the electrochemically active bacteria, or perform some other desirable function.

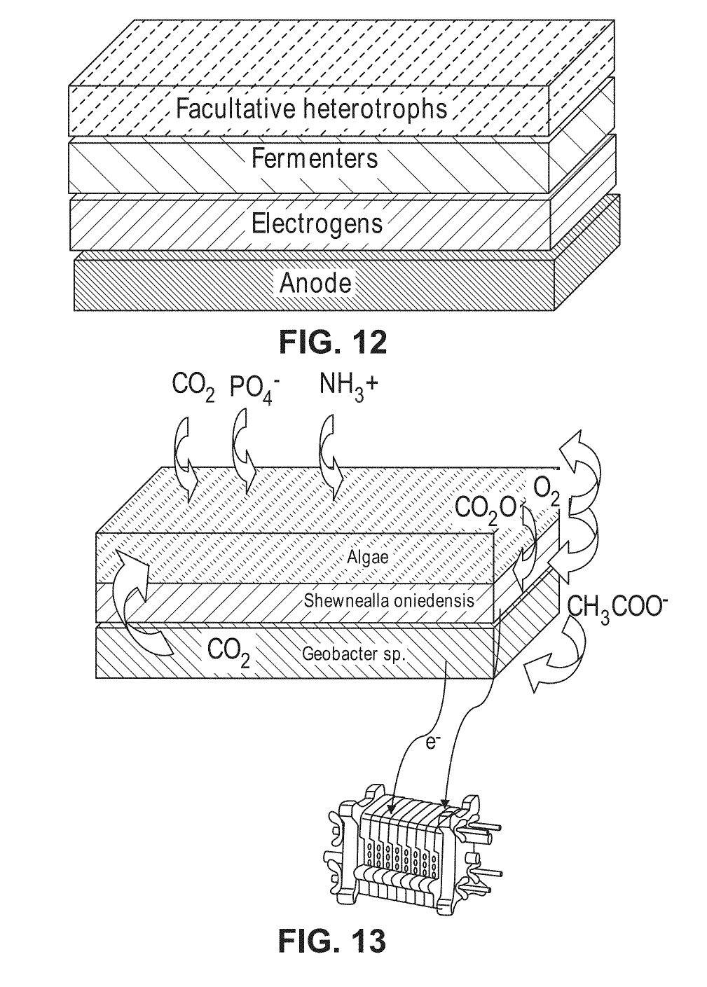

As a more specific example, the present disclosure provides an MFC incorporating an artificial biofilm comprising multiple modular microbial communities which, as a whole, are able to simultaneously remove organic and inorganic contaminants from waste water to produce reusable water while creating electricity or hydrocarbon fuel. For the purposes of the present disclosure, the term “reusable water” is intended to mean water suitable for drinking, irrigation and commercial purposes. A general depiction of the artificial biofilm is shown in FIG. 12.

In the depicted embodiment, a biofilm comprising three modules is positioned on the anode of an MFC that is fed waste water. The first module contains encapsulated facultative heterotrophs such as Pseudomonas sp. and α-Proteobacteria. The facultative heterotrophs metabolize pesticides in the environment (i.e. in waste water). The second module contains encapsulated fermenters such as Clostridia and Bacteriodetes. The fermenters metabolize organic compounds in the environment to produce gas and small organic acids. The third module contains encapsulated electrogens such as Shewanella oneidensis, Geobacter sulfurreducens, Shewanella putrefaciens, or Aeromaonas hydrophila. The electrogens use the products of the fermentative organisms as fuel for CO₂, electron and proton generation. As stated above, the MFC then combines the electrons and protons generated by the electrogens with oxygen to produce clean water. A further advantage of the artificial biofilms disclosed herein is demonstrated by the immunity of these system to population fluctuation, the presence of other solids, and/or competing bacteria that frequently plague phosphate removal, in particular, enabling the MFCs to be relatively stable and long-lived.

According to another embodiment, dissimilatory metal reducing bacteria (DMRB) such as Shewanella oneidensis are encapsulated in a first module while algae is encapsulated in another module. DMRB are able to remove a plurality of organic compounds, including pesticides and inorganic compounds such as ammonia, phosphate and heavy metals from waste water Immobilized algae in the second module removes heavy metals, phosphorous and nitrogen. Together they are able to produce clean water.

According to an embodiment of the present disclosure, shown in FIG. 13, the DMRB/algae system described above is combined with, for example, Geobacter sp. in a third module, so as produce a self-regenerating carbon neutral electrical system.

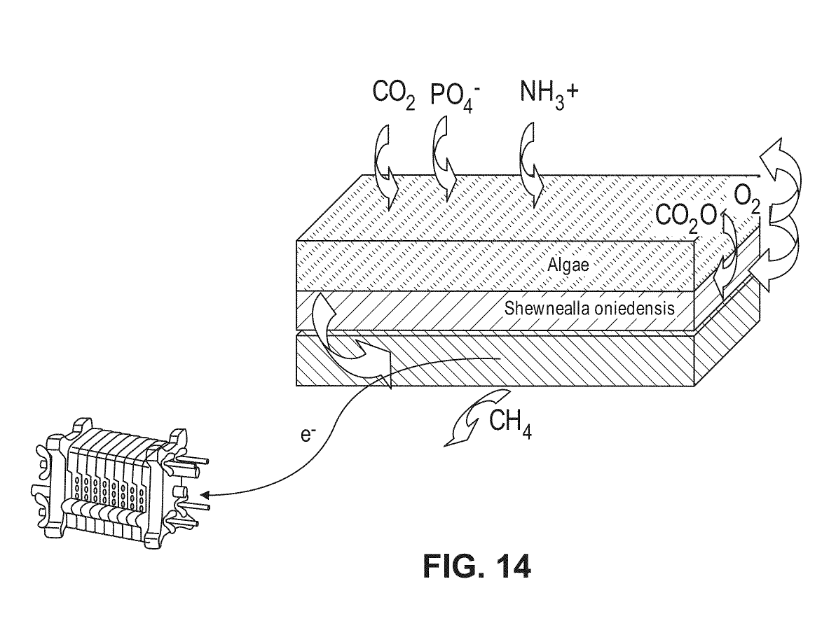

Yet another alternative example is shown in FIG. 14, where the Geobacter sp. is replaced with a syntrophic acetate-methanogenic community, which generates methane which can then be collected for power use.

Additional organisms that could be encapsulated include, but are not limited to, Acinetobacter venetianus to remove oil spills, and Pseudomonas spp to remove pesticides. In each case, as shown in FIGS. 2-4, the biofilms of the present disclosure are physically and/or electrically connected to an electrode, which can then produce energy for a variety of applications.

According to a specific embodiment, it is anticipated that the MFC described herein could be a small, portable self-contained unit that could be used, for example, in rural locations to generate clean water and electricity from waste water.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise.

References: All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications. Additional relevant disclosure and/or information may be found in Han W, Ista L K, Gupta G, Li L, Harris J M, and López G P., in Handbook of Nanomaterials Properties, Bushan, B. ed. Springer-Verlag GmbH, Heidelberg, 2014, which is incorporated by reference, as well as in the following:

Avnir, D., Braun, S., Lev, O. and Ottolenghi, M. Enzymes and Other Proteins Entrapped in Sol-Gel Materials Chemistry of Materials 1994 6 (10), 1605-1614

Brennan, L. & Owende, P. Biofuels from microalgae—A review of technologies for production, processing, and extractions of biofuels and co-products. Renewable and Sustainable Energy Reviews 14, (2010).

de-Bashan, L., Bashan, Y., Moreno, M., Lebsky, V. & Bustillos, J. Increased pigment and lipid content, lipid variety, and cell and population size of the microalgae Chlorella spp. when co-immobilized in alginate beads with the microalgae-growth-promoting bacterium Azospirillum brasilense. Canadian journal of microbiology 48, 514-21 (2002).

Dickson, D. & Ely, R. Evaluation of encapsulation stress and the effect of additives on viability and photosynthetic activity of Synechocystis sp. PCC 6803 encapsulated in silica gel. Applied microbiology and biotechnology 91, 1633-46 (2011).

Dickson, D. & Ely, R. Silica sol-gel encapsulation of cyanobacteria: lessons for academic and applied research. Applied microbiology and biotechnology 97, 1809-19 (2013).

Fennouh, S., Guyon, S., Livage, J. & Roux, C. Sol-gel entrapment of Escherichia coli. Journal of Sol-Gel Science and Technology 19, 647-649 (2000).

Ferrer, M. L., Yuste, L., Rojo, F. & Monte, F. del Biocompatible Sol-Gel Route for Encapsulation of Living Bacteria in Organically Modified Silica Matrixes. Chemistry of Materials 15, (2003).

Gonzalez, L. E. & Bashan, Y. Increased Growth of the Microalga Chlorella vulgaris when Coimmobilized and Cocultured in Alginate Beads with the Plant-Growth-Promoting Bacterium Azospirillum brasilense. Applied and Environmental Microbiology 66, (2000).

Gonzalez-Bashan, L., Lebsky, V., Hernandez, J., Bustillos, J. & Bashan, Y. Changes in the metabolism of the microalga Chlorella vulgaris when coimmobilized in alginate with the nitrogen-fixing Phyllobacterium myrsinacearum. Canadian journal of microbiology 46, 653-9 (2000).

Lebsky, V. K., Gonzalez-Bashan, L. E. & Bashan, Y. Ultrastructure of interaction in alginate beads between the microalga Chlorella vulgaris with its natural associative bacterium Phyllobacterium myrsinacearum and with the plant growth-promoting bacterium Azospirillum brasilense. Canadian Journal of Microbiology 47, (2001).

Liu, K., Li, J., Qiao, H., Lin, A. & Wang, G Immobilization of Chlorella sorokiniana GXNN 01 in alginate for removal of N and P from synthetic wastewater. Bioresource technology 114, 26-32 (2012).

Pierre, A. C. The sol-gel encapsulation of enzymes. Biocatalysis and Biotransformation 22, (2004).

Pope, E. J., Braun, K. & Peterson, C. M. Bioartificial organs I: silica gel encapsulated pancreatic islets for the treatment of diabetes mellitus. Journal of Sol-Gel Science and Technology 8, 635-639 (1997).

Rooke, J. C. et al. Novel photosynthetic CO2 bioconvertor based on green algae entrapped in low-sodium silica gels. Journal of Materials Chemistry 21, (2011).

Wang, B., Zhang, J. & Dong, S. Silica sol-gel composite film as an encapsulation matrix for the construction of an amperometric tyrosinase-based biosensor. Biosensors & bioelectronics 15, 397-402 (2000).

What is claimed is:

1. An artificial biofilm comprising a metabolically active and stable, encapsulated population or community of multiple types of microorganisms or cells.

2. The artificial biofilm of claim 1 wherein the community is encapsulated in a silica-based gel.

3. The artificial biofilm of claim 1 wherein the encapsulated population comprises a bacteria, algae, or cyanobacterium.

4. The artificial biofilm of claim 1 comprising multiple modules wherein each module comprises at least one type of encapsulated microorganism or cell.

5. The artificial biofilm of claims 4 wherein a first encapsulated microorganism is bacteria.

6. The artificial biofilm of claim 4 wherein the second encapsulated organism comprises eukaryotic cells.

7. The artificial biofilm of claim 5 wherein the bacteria is electrochemically active.

8. The artificial biofilm of claim 7 wherein the bacteria is selected from the group consisting of Shewanella oneidensis, Geobacter sulfurreducens, Shewanella putrefaciens, or Aeromaonas hydrophila.

9. The artificial biofilm of claim 5 wherein the bacteria is a dissimilatory metal reducing bacteria.

10. The artificial biofilm of claim 9 wherein the bacteria is Shewanella oneidensis.

11. The artificial biofilm of claim 4 wherein a first encapsulated microorganism is algae.

12. The artificial biofilm of claim 5 wherein a second encapsulated microorganism is algae.

13. The artificial biofilm of claims 5 wherein a second encapsulated microorganism is a cyanobacterium.

14. The artificial biofilm of claims 5 wherein a second encapsulated microorganism is a heterotroph.

15. The artificial biofilm of claim 12 wherein the first encapsulated microorganism is a dissimilatory metal reducing bacteria, and a third encapsulated microorganism is a second dissimilatory metal reducing bacteria or an acetate/methanogen syntroph.

16. A method for producing electricity and reusable water from waste water comprising: exposing the waste water to a microbial fuel cell comprising an electrode comprising artificial biofilm comprising a metabolically active and stable, encapsulated population or community of multiple types of microorganisms or cells.
